(19) United States
(12) Patent Application Publication
(10) Pub. No.: US 2014/0277408 A1
(43) Pub. Date: Sep. 18, 2014

(54) Prosthetic Heart Valve System

(71) Applicant: Boston Scientific Scimed, Inc., Maple Grove, MN (US)

(72) Inventor: Martyn G. Folan, Loughrea (IE)

(73) Assignee: BOSTON SCIENTIFIC SCIMED, INC., Maple Grove, MN (US)

(21) Appl. No.: 14/202,870

(22) Filed: Mar. 10, 2014

Related U.S. Application Data
(60) Provisional application No. 61/777,505, filed on Mar. 12, 2013.

(51) Int. Cl. A61F 2/24

(52) U.S. Cl. CPC A61F 2/2412; A61F 2/2436
USPC 623/2.11; 623/2.12

(57) ABSTRACT

In at least one embodiment, a prosthetic valve assembly comprises a pouch connecting two valves to one another. The valve assembly is delivered by a catheter system. In some embodiments, the valve assembly includes a valve for replacing a mitral valve and a valve for replacing an aortic valve. The valve assembly may optionally include an adhesive layer on the outer surface.

### CROSS-REFERENCE TO RELATED APPLICATIONS

This application claims priority to U.S. Patent Provisional Application No. 61/777,505, filed Mar. 12, 2013, the entire contents of which are hereby incorporated herein by reference.

### STATEMENT REGARDING FEDERALLY SPONSORED RESEARCH

Not Applicable

### BACKGROUND OF THE INVENTION

Valve replacement may be indicated when there is a narrowing of the native valve, commonly referred to as stenosis, or when the native valve leaks or regurgitates. Examples of valves that may be replaced include heart valves and venous valves. Valve replacement is used to repair or replace a diseased valve. Currently ten percent of patients with valvular heart disease have involvement of both aortic and mitral valves. To replace a heart valve, one method is open heart surgery. Another method to replace a heart valve, typically aortic valve, is transcatheter aortic valve implantation (TAVI), developed to treat high-risk or inoperable severe aortic stenosis patients, which is known to have a poor prognosis clinically.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, a prosthetic valve assembly comprises a pouch connecting two valves to one another. The valve assembly is delivered by a catheter system. In some embodiments, the valve assembly includes a valve for replacing a mitral valve and a valve for replacing an aortic valve. The valve assembly may optionally include an adhesive layer on the outer surface.

These and other embodiments which characterize the invention are pointed out with particularity in the claims annexed hereto and forming a part hereof However, for further understanding of the invention reference can be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1 is partial cross-sectional view of a heart.

FIGS. 2A-C show blood flow through the left chambers of the heart.

FIG. 3 is a schematic view of a valve.

FIG. 4 is a schematic view of a delivery system for the valve of FIG. 3.

FIG. 5 is a detailed view of an embodiment of the delivery system of FIG. 4.

FIG. 6a is a cross-sectional view of the delivery system of FIG. 5 at line A-A shown prior to deployment.

FIG. 6b is a cross-sectional view of the delivery system of FIG. 5 at line A-A shown following deployment.

FIG. 7a is a cross-sectional view of the delivery system of FIG. 5 at line B-B shown prior to deployment.

FIG. 7b is a cross-sectional view of the delivery system of FIG. 5 at line B-B shown following deployment.

FIG. 8a is a cross-sectional view of the delivery system of FIG. 5 at line C-C shown prior to deployment.

FIG. 8b is a cross-sectional view of the delivery system of FIG. 5 at line C-C shown following deployment.

FIGS. 9-15 schematically show a method of implanting the valve of FIG. 3.

FIG. 16 shows an alternative embodiment to the fully deployed valve assembly shown in FIG. 15.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

FIG. 1 is a cross-sectional view of a heart 10 that has four chambers, a left atrium 12, a left ventricle 14, a right atrium 16 and the right ventricle 18. The heart 10 has four valves, an aortic valve 20, a mitral valve 22, a pulmonary valve 24, and a tricuspid valve 26. In the left side of the heart 10, blood flows from the left atrium 12 to the left ventricle 14, and then exits the heart 10 through the aorta 28. In the right side of the heart 10, blood flows from the right atrium 16 to the right ventricle 18 and out of the heart 10 through the pulmonary artery 30.

FIGS. 2A-C show how blood flows through the left chambers of the heart. FIG. 2A shows blood volume in the left atrium 12 being retained by a closed mitral valve 22. FIG. 2B shows systole contraction (arrows) of the left atrium 12 with a closed aortic valve 20, which pumps blood into the left ventricle 14. FIG. 2C shows systole contraction (arrows) of the left ventricle 14 with a closed mitral valve 22 and an open aortic valve 20, which pumps blood out of the left ventricle 14 into the aorta 28.

FIG. 3 is a schematic view of a prosthetic valve assembly 40 comprising a first valve 42, a second valve 44, and a pouch 46 connecting the first and second valves 42, 44. As used in this application, the terms “connect” or “engage” do not include “indirect” connection or engagement. Thus, for example Element B “connecting” Elements A and C, directly connects A and C with no other element between A and B or between B and C. Any suitable means can be used to connect the ventricular pouch 46 to the valves 42, 44. In some embodiments, an adhesive is used to connect the pouch 46 to the valves 42, 44. Any suitable adhesive may be used to connect the pouch 46 to the valves 42, 44. In other embodiments, the pouch 46 is sutured to the valves 42, 44.

In at least one embodiment, the valve assembly 40 is a heart valve and the pouch 46 is a ventricular pouch. In some embodiments, the valve assembly 40 provides for an integrated dual valve replacement in the heart. In at least one embodiment, the prosthetic valve eliminates surgical intervention for heart valve repair.

In some embodiments, the first valve 42 is a prosthetic mitral valve, the second valve 44 is a prosthetic aortic valve, and the pouch 46 is sized for the left ventricle of a heart. In other embodiments, the first valve 42 is a prosthetic tricuspid valve and the second valve 44 is a prosthetic pulmonary valve. In some embodiments, the valve assembly 40 eliminates valvular regurgitation. In other embodiments, the valve assembly 40 eliminates valve displacement. Without being bound by theory, the ventricular pouch acts as a stabilizing component for the valve when the valve is implanted in the heart.

In at least one embodiment, the valves 42, 44 are self-expanding. In some embodiments, each valve 42, 44 has at least two valve leaflets. In one embodiment, the first valve 42 has two valve leaflets and the second valve has three valve leaflets. In at least one embodiment, the first valve 42 regulates flow into the pouch 46 and the second valve 44 regulates flow out of the pouch 46.

Valves suitable for the first and second valves 42, 44 include, but are not limited to, valves disclosed in the following publications, hereby incorporated by reference herein in their entireties: WO 2005/062980; US 2007/0129788; US 2008/0319526; US 2009/0171456; US 2009/0306768; US 2009/0030512; US 2011/0060405; US 2012/0179239; US 2013/0035758; U.S. Pat. No. 8,070,802; U.S. Pat. No. 7,892,276; U.S. Pat. No. 7,780,722; U.S. Pat. No. 7,670,368; U.S. Pat. No. 7,566,343; U.S. Pat. No. 8,012,198; U.S. Pat. No. 6,685,739; U.S. Pat. No. 7,569,071; U.S. Pat. No. 7,867,274; U.S. Pat. No. 7,776,053; U.S. Pat. No. 7,722,666; U.S. Pat. No. 8,128,681; U.S. Pat. No. 7,416,557; valves disclosed in Collapsible Caged-Ball Prosthetic Valve for Transcatheter Delivery and Method of Use, Attorney Docket Number S63.2C-15620-US01, with inventors Huisun Wang and Pu Zhou; valve disclosed in Replacement Heart Valve, Attorney Docket Number S63.2C-15619-US01, with inventors Roger Hastings and Mark Jenson. Additional valves suitable for the first and second valves 42, 44 include valves developed by Boston Scientific Scimed, such as the Lotus Aortic Valve System; valves developed by Edwards, such as the Sapien Transcatheter Heart Valve; valves developed by CardiAQ™ Valve Technologies, such as the CardiAQ prosthesis; valves developed by St. Jude Medical, such as the Trifecta™ valve, the Regent™ aortic valve; and valves developed by Medtronic, such as the Core Valve®.

The valves 44, 46 may be made from any suitable biocompatible materials including one or more polymers, one or more metals, or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The valves 44, 46 may be made of shape memory materials such as superelastic Nitinol or spring steel, or may be made of materials which are plastically deformable. In the case of shape memory materials, the valves 44, 46 may be provided with a memorized shape and then deformed to a reduced diameter shape. The valves 44, 46 may restore itself to its memorized shape upon being heated to a transition temperature and having any restraints removed therefrom.

The valves 44, 46 may be created by methods including cutting or etching a design from a tubular stock, from a flat sheet which is cut or etched and which is subsequently rolled; or from one or more interwoven wires or braids. Any other suitable technique which is known in the art or which is subsequently developed may also be used to manufacture the valves 44, 46.

In at least one embodiment, the pouch 46 includes a first opening 52 and a second opening 54. In some embodiments, the first opening 52 has a diameter equal to the expanded diameter of the first valve 42 and the second opening 54 has a diameter equal to the expanded diameter of the second valve 44. In at least one embodiment, the circumference of the first opening 52 of the pouch 46 is attached to the circumference of an end of the first valve 42 and the circumference of the second opening 54 of the pouch 46 is attached to the circumference of an end of the second valve 44. Thus, the pouch 46 is connected to an end of each valve 42, 44. This embodiment for example provides for a closed system with fluid flowing only through the prosthetic heart valve assembly 40 from the left atrium 12 to the aorta 28.

In at least one embodiment, the main compartment 56 of the pouch 46 includes a first span 48 and a second span 50. In at least one embodiment, the second span 50 has a greater length than the first span 48. In some embodiments, the main compartment 56 of the pouch 46 has a size or volume that approximates the size or volume of a heart ventricle 14, 18. This is shown for example in FIG. 15. As used herein the “main compartment” of the pouch is the portion of the pouch that extends between the two valves 42, 44. In other embodiments, the size or volume of the main compartment 56 of the pouch 46 is less than the size or volume of a heart ventricle 14, 18. Thus this pouch 46 is a reduced volume ventricular pouch 46. A reduced volume ventricular pouch 46 is shown for example in FIG. 16. In at least one embodiment a pouch 46 having a main compartment 56 with a size or volume that is less than the size or volume of a heart ventricle 14, 18 is used to treat cardiomyopathy. Without being bound by theory, a ventricular pouch having a main compartment 56 with a size or volume that is less than the size or volume of the heart ventricle reduces the volume of blood that is ejected with each ventricle systole action which reduces the pressure on weakened heart muscle to function at a capacity to execute efficient blood ejection from the ventricle.

In at least one embodiment, the pouch 46 at least partially lines the valve 42, 44 (not shown). In this embodiment, portions of the pouch 46 function as the liner for the valve instead of having a separate liner for each valve. In some embodiments, the pouch has a first tubular section extending from the first opening 52 to the main compartment 56 of the pouch where the first tubular section forms an outer liner attached to the outer surface of the first valve 42, and a second tubular section extending from the second opening 54 to the main compartment 56 of the pouch where the second tubular section forms an outer liner of the second valve 44. In one embodiment, the liners help prevent leakage between the valves 42, 44 and the lumen wall.

In at least one embodiment, the pouch is tubular (not shown). In some embodiments, a valve with a tubular pouch is used to replace two native valves, for example two venous valves. In one embodiment, the tubular pouch is a graft and any suitable graft material can be used.

In at least one embodiment, the ventricular pouch 46 restricts or severs existing connective structures, such as papillary muscle, chordae tendineae, between the valvular components and the native heart. In some embodiments, the valve assembly 40 has an annulus positioned lower than the native valve annulus to encourage a disconnection of the connective tissue that are present at the native valve (not shown). In at least one embodiment, one or both of the valves 42, 44 has a protruding lip at the valve end which is attached to the pouch 46, the lowest extremity of the valve 42, 44 (not shown). When this valve assembly 40 embodiment is implanted, the protruding lip aids securement in situ and also causes a disruption or severing of the connective tissue. Even if the protruding lip of the implanted valve does not sever the connective tissue, the contractions of the heart muscle that cause the tendons to pull open the native valve are not effective because the prosthetic valve interrupts this tension. Thus, when the valve is implanted these connective tendons are essentially redundant and trapped between native heart wall and the valve.

In at least one embodiment, the inner wall of the ventricular pouch includes prosthetic attachments that are configured to mimic the native connective structures such as papillary muscle and chordae tendineae (not shown). In some embodiments, a system of prosthetic chordae tendineae or retaining wires that mimic native chordae tendinaea are connected to the inner surface of pouch in a similar fashion as that of the native system.

In at least one embodiment, the pouch 46 is made of a polymeric material. Suitable polymeric materials for the pouch 46, include but are not limited to, polyurethane and its copolymers, silicone and its copolymers, ethylene vinyl-acetate, polyethylene terephthalate (PET), thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polycarbonate, poly(glycolide-lactide) copolymer, Tecothane, PEBAX®, polyethylene, polylactic acid, poly(γ-caprolactone), poly(γ-hydroxybutyrate), polydioxanone, poly(γ-ethyl glutamate), polyiminocarbonates, poly(ortho ester), and/or polyanhydrides. Additional polymeric materials are described, for example, in U.S. Pat. Nos. 5,650,234 and 5,463,010, herein incorporated in their entirety. Blends of the above polymers may also be employed.

In at least one embodiment, the outer surface of the pouch 46 of the valve assembly 40 has an adhesive 60 thereon, as shown for example in FIG. 3. In some embodiments, the entire outer surface of the pouch 46 has a layer of adhesive 60. In at least one embodiment, the adhesive material has an affinity for adherence to the inner surface of the ventricle 14, 18 so that after contact between the ventricular pouch and the ventricle the ventricular pouch adheres to the inner surface of the ventricle 14, 18.

Any suitable bioadhesive material may be employed herein and can include natural polymeric materials, as well as synthetic materials, and synthetic materials formed from biological monomers such as sugars. Bioadhesives can also be obtained from the secretions of microbes or by marine mollusks and crustaceans. Bioadhesives are designed to adhere to biological tissue. In at least one embodiment, the adhesive activity of the adhesive layer is controlled through compound design such that an exposure time is required for tracking the device to the heart before the adhesive is ready to bond to the ventricle or other lumen wall.

Examples of bioadhesives include, but are not limited to, amino adhesives, adhesive surface proteins (MSCRAMMS), adhesively modified biodegradable polymers such as Fatty Ester Modified PLA/PLGA, polymer materials, minigel particles, each discussed in detail below, as well as mixtures thereof.

Suitably, the bioadhesive is dissolved in a solvent or cosolvent blend prior to application. Suitable solvents include, but are not limited to, alcohols including methanol, ethanol and isopropanol, and water.

The following examples of bioadhesives are intended for illustrative purposes only, and not as a limitation on the scope of the present disclosure.
1. Amino Acids: Amino acids can be utilized to facilitate adhesion to the lesion site. Zwitterionic amino acids can be employed as a layer or as a component within pouch layer. The zwitterionic amino acid can be oriented so that the hydrophobic side of the zwitterionic amino acid selectively facilitates adhesion to the lipophilic hearty wall. One example of a useful compound is amino acid 3,4-L-dihydroxyphenylalanine (DOPA), a tyrosine derivative found in high concentrations in the “glue” proteins of mussels.
2. Adhesive Surface Proteins: Protein adhesions called MSCRAMMs (microbial surface components recognizing adhesive matrix molecules) can also be employed as a bioadhesive in the second coating composition. MSCRAMMS are naturally produced by pathogens to initiate adhesion to the host extra cellular matrix to initiate infection. These adhesive surface proteins can be isolated or synthesized and utilized as a separate layer to facilitate adhesion the lesion site.
3. Adhesively Modified Biodegradable Polymers: One example of an adhesively modified biodegradable polymer is a DOPA (L-3,4-dihydroxyphenylalanine) modified PLA (polylactic acid), or PLGA poly(lactide-co-glycolide). In this embodiment, examples of suitable adhesive moieties include, but are not limited to, monopalmitate, monostearin, glycerol, and dilaurin or iso-stearyl alcohol.
4. Polymer Materials: Proteins such as gelatin and carbohydrates such as starch may also be employed. Polysaccharides such as sorbitol, sucrose, xylitol, anionic hydrated polysaccharides such as gellan, curdlan, XM-6, and xanthan may also be employed as a bioadhesive. Others include derivatives of natural compositions such as algenic acid, hydrated gels and the like, and also biocompatable polymers and oligomers such as dextrans, dextranes and dextrins, hydrogels including, but not limited to, polyethylene glycol (PEG), polyethylene glycol/dextran aldehyde, polyethylene oxide, polypropyline oxide, polyvinylpyrrolidine, polyvinyl acetate, polyhydroxyethyl methacrylate and polyvinyl alcohol, as well as derivatives thereof may also be employed herein. See for example U.S. Pat. No. 6,391,033 and Aldehyde-Amine Chemistry Enables Modulated Biosealants with Tissue-Specific Adhesion by Artzi et al. (Advanced Materials Vol. 21, Issue 32-33, pages 3399-3403) the entire content of each is incorporated by reference herein.
5. Minigel Particles: Another bioadhesive is poly(NIPAM) (poly(N-isopropylacrylamide) minigel particles. This polymer has the property of being in a liquid state at room temperature and an adhesive at body temperature, see “Preparation and Swelling Properties of Poly(NIPAM) “Minigel” Particles Prepared by Inverse Suspension Polymerization”, Dowding, John et al., Journal of Colloid and Interface Science 221, 268-272 (2000), available online at http:/www.idealibrary.com, the entire content of which is incorporated by reference herein.

For better retention of the polymer on the pouch surface, several techniques may be employed. Suitably, the minigel particles are crosslinked or mixed with a higher molecular weight polymer to allow enough time for retention of the minigel to the medical device during delivery, or uncrosslinked minigel particles can be employed in a crosslinked polymer network.

In at least one embodiment, the valve assembly 40 is delivered by a catheter. In some embodiments, the valve assembly 40 is delivered by a delivery system suitable for transcatheter aortic valve implantation (TAVI). FIG. 4 is a schematic drawing of a delivery system 80 for the valve assembly 40 positioned on a delivery catheter 82. In some embodiments, the delivery system 80 includes a catheter 82 has a tip 84 and defines a guidewire lumen 88 for a guidewire 100.

In some embodiments, the delivery system 80 includes a sheath 86 which maintains the valve assembly 40 in a delivery configuration, as is known in the art for the delivery of self-expanding prostheses such as stents and valves. For example, as shown in FIG. 4, the valve assembly 40 is positioned around a catheter shaft 83 and a sheath 86 is positioned around the valve assembly 40. The sheath 86 can be made of any suitable material. In some embodiments, the sheath 86 is a polymeric sheath. In other embodiments, the delivery system 80 includes a first balloon and a second balloon (not shown).

In at least one embodiment, the pouch 46 has a folded delivery configuration. In some embodiments, the sheath 86 maintains the pouch 46 in the folded delivery configuration. FIG. 7 shows an example of the pouch 46 in a folded delivery configuration. In some embodiments, the pouch has a plurality of circumferential folds 91 longitudinally adjacent one another. In other embodiments, the pouch has a plurality of longitudinal folds 91.

In at least one embodiment, such as is shown in FIGS. 5-8b, the catheter shaft 83 has a variable diameter. In other embodiments, the catheter defines a notch or cavity 90. The cavity 90 is defined by a section 96 of the catheter shaft 83 which has a reduced diameter relative to the proximally and distally adjacent sections 92 and 96. The cavity 90 of the catheter 82 accommodates the folded delivery configuration of the pouch 46.

In some embodiments the section 96 of the catheter shaft 83 positioned within the lumen of the second valve 44 has a first diameter; the section 94 of the catheter shaft 83 positioned within the lumen of the ventricular pouch 46 has a second diameter; and the section 92 of the catheter shaft 83 positioned within the lumen of the first valve 42 has a third diameter where the second diameter is less than the first diameter and less than the third diameter. In at least one embodiment, the first diameter is equal to the third diameter. In some embodiments, the first and third diameters are equal to the diameter of a 15F delivery catheter and the second diameter is equal to the diameter of an 8F delivery catheter. In some embodiments, the second diameter portion of the catheter shaft 83 accommodates the folds of the pouch 46 for delivery.

As is depicted in FIG. 7a, in some embodiments, the cavity 90 accommodates the folds 91 of the pre-delivered or reduced diameter pouch 46 for delivery. Upon deployment of the pouch 46 (discussed in greater detail below) the pouch 46 unfolds in the manner shown in FIG. 7b as it expands or “falls” into the ventricle cavity.

In at least one embodiment, the section 96 and section 92 of the catheter shaft 83 each include a balloon for expanding the first valve and the second valve (not shown).

FIGS. 9-15 schematically show delivery of the valve assembly 40. Although reference is made to delivering the valve assembly 40 to the left ventricle 14, the valve assembly 40 could also be delivered to the right ventricle 18 or to another suitable location in the vasculature.

FIG. 9 shows advancement of the guidewire 100 through the aorta 28, into the left ventricle 14 and into the left atrium 12 (arrows show the flow of blood). FIGS. 10-11 show the advancement of the delivery system 80 over the guidewire 100 until the first valve 42 is positioned inside the left atrium 12. FIGS. 9-11 show nominal pulsatile blood capacity and blood movement (arrows)

FIG. 12 shows expansion of the first valve 42. In some embodiments, withdrawal of the sheath 86 (See FIG. 4) provides for self-expansion of the first valve 42. In other embodiments, the first valve 42 is expanded by the first balloon. In at least one embodiment, the first valve 42 expands to sit in the mitral valve cavity. In some embodiments, the first valve 42 seals off the native valve annulus to prevent flow into the native ventricle. Thus, the blood contents of the left atrium 12 are isolated from the left ventricle 14. After expansion of the first valve 42, the ventricle has residual pre-valve expansion blood capacity and movement of the residual blood is shown by arrows. In a least one embodiment, each left ventricle contraction (systole) after expansion of the first valve 42 provides for the expulsion of the residual blood volume which provides for space for the pouch 46 to expand.

FIGS. 13-14 show expansion of the pouch 46 from the folded delivery configuration after retraction of the sheath. In at least one embodiment, blood expelled from the left atrium 12 fills the pouch 46 (arrows indicate the contraction of the left atrium). Upon expansion of the pouch 46, the external adhesive 60 provides for adherence of the pouch 46 to the inner surface of the left ventricle 14. In some embodiments, the pouch 46 contacts the inner surface of the left ventricle 14 due to the pressure provided by blood inside the pouch 46. In at least one embodiment, liquid in the left ventricle 14 activates the adhesive 60.

Upon expansion of the pouch 46, the second valve 44 is expanded, as shown for example in FIG. 15. In some embodiments, the second valve 44 expands upon retraction of the sheath 86. In other embodiment, the second valve 44 is expanded by the second balloon (not shown). Thus, the valve assembly 40 is sequentially deployed with the first valve 42 being deployed before the pouch 46 and the pouch being deployed before the second valve 44.

In at least one embodiment, after expansion of the second valve 44, the valve assembly 40 is deployed in the heart and blood flows from the left atrium 12 to the aorta 28 via the valve assembly 40.

As mentioned previously, in some embodiments (an example of which is shown in FIG. 15) when the pouch 46 is fully deployed, the pouch 46 will extend entirely within and fully occupy the ventricle space. Alternatively, in some embodiments (an example of which is shown in FIG. 16) the pouch 46 has a reduced size and/or volume such that the fully deployed pouch 46 occupies only a portion of the ventricle space.

In some embodiments the valve assembly 40, the delivery system 80, or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the valve assembly 40 is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof Where the therapeutic agent includes a polymer agent, the polymer agent may be a polystyrene-polyisobutylene-polystyrene triblock copolymer (SIBS), polyethylene oxide, silicone rubber and/or any other suitable substrate.

The following non-limiting numbered statements are directed to one or more aspects of the valve assembly 40, delivery system 80 for the valve assembly 40, or delivering the valve assembly 40:
1. A valve assembly comprising:

a first valve;

a second valve;

a pouch connecting the first and second valve.
2. The valve assembly of statements 1, the first valve comprising a first leaflet and a second leaflet; and

the second valve comprising a first leaflet, a second leaflet, and a third leaflet.
3. The valve assembly of statement 1-2, further comprising a layer of adhesive on an outer surface of the prosthetic valve.
4. The valve assembly of statements 1-3, the pouch comprising a main compartment having a maximum size and volume equal to a size and volume of a heart ventricle.
5. The valve assembly of statements 1-4, wherein the first valve is configured to replace a native mitral valve and the second valve is configured to replace a native aortic valve.
6. The valve assembly of statements 1-5, wherein the first valve is configured to regulate flow of blood into the pouch, and the second valve is configured to regulate flow of blood out of the pouch.
7. The valve assembly of statements 1-3 and 6, the pouch being tubular.
8. The valve assembly of statements 1-3 and 5-7, the pouch comprising:

a main compartment;

a first tubular section extending from the main compartment, the first tubular section attached to the outer surface of the first valve; and

a second tubular section extending from the main compartment, the second tubular section attached to the outer surface of the second valve.
9. A delivery system comprising:

a catheter comprising a sheath defining a lumen and a shaft positioned inside the lumen of the sheath;

a valve assembly, the valve assembly positioned around the shaft and inside the sheath, the valve assembly comprising a first valve, a second valve, and a pouch connecting the first and second valves.
10. The delivery system of statement 9, the shaft of the catheter having a first section with a first diameter, a second section with a second diameter, and a third section with a third diameter where the second diameter is less than the first diameter rand the second diameter is less than the third diameter;

wherein the first valve is positioned around the first section, the pouch is positioned around the second section, and the second valve is positioned around the third section.
11. The delivery system of statements 9-10, wherein the pouch has a plurality of folds.
12. The delivery system of statement 11, wherein the plurality of folds are circumferential folds.
13. The delivery system of statement 11, wherein the plurality of folds are longitudinal folds.
14. The delivery system of statements 9-13, the shaft defining a guidewire lumen extending from a proximal end region of the shaft to a distal end of the shaft.
15. The delivery system of statements 9-14, the first valve comprising a first leaflet and a second leaflet; and

the second valve comprising a first leaflet, a second leaflet, and a third leaflet.
16. The delivery system of statement 9-15, the valve assembly assembly further comprising a layer of adhesive on an outer surface of the valve assembly.
17. The delivery system of statements 9-16, wherein the first valve is configured to replace a native mitral valve and the second valve is configured to replace a native aortic valve.
18. The delivery system of statements 9-17, wherein the first valve is configured to regulate flow of blood into the pouch, and the second valve is configured to regulate flow of blood out of the pouch.
19. The delivery system of statements 9-16 and 18, the pouch being tubular.
20. The delivery system of statements 9-19, the pouch further comprising:

a main compartment;

a first tubular section extending from the main compartment, the first tubular section attached to the outer surface of the first valve; and

a second tubular section extending from the main compartment, the second tubular section attached to the outer surface of the second valve.
21. The delivery system of statements 9-18 and 20, the pouch comprising a main compartment having a maximum size and volume equal to a size and volume of a heart ventricle.
22. A method of delivering a valve assembly comprising deploying a valve at a desired location in a body lumen, the valve comprising a first valve, a second valve, and a pouch connecting the first and second valves.
23. The method of statement 22, wherein deploying comprises sequential deployment wherein the first valve is expanded before the pouch and the pouch is expanded before the second valve.
24. The method of statements 22-23, wherein deploying comprises advancing a catheter comprising a sheath defining a lumen and a shaft positioned inside the lumen of the sheath wherein the valve is positioned around the shaft and inside the sheath.
25. The method of statement 24, the shaft of the catheter having a first section with a first diameter, a second section with a second diameter, and a third section with a third diameter where the second diameter is less than the first diameter rand the second diameter is less than the third diameter;

wherein the first valve is positioned around the first section, the pouch is positioned around the second section, and the second valve is positioned around the third section.
26. The method of statements 24-25, wherein the pouch has a plurality of folds.
27. The method of statement 26, wherein the plurality of folds are circumferential folds.
28. The method of statement 26, wherein the plurality of folds are longitudinal folds.
29. The method of statements 24-28, the shaft defining a guidewire lumen extending from a proximal end region of the shaft to a distal end of the shaft.
30. The method of statements 22-29, the first valve comprising a first leaflet and a second leaflet; and

the second valve comprising a first leaflet, a second leaflet, and a third leaflet.
31. The method of statement 22-30, the valve further comprising a layer of adhesive on an outer surface of the prosthetic valve.
32. The method of statements 22-31, the pouch comprising a main compartment having a maximum size and volume equal to a size and volume of a heart ventricle.
33. The method of statements 22-32, wherein the first valve is configured to replace a native mitral valve and the second valve is configured to replace a native aortic valve.
34. The method of statements 22-33, wherein the first valve is configured to regulate flow of blood into the pouch, and the second valve is configured to regulate flow of blood out of the pouch.
35. The method of statements 22-31, the pouch being tubular.
36. The method of statements 22-35, the pouch further comprising:

a first tubular section extending from the main compartment, the first tubular section attached to the outer surface of the first valve; and

a second tubular section extending from the main compartment, the second tubular section attached to the outer surface of the second valve.
37. The method of statements 22-36, wherein the first valve is self-expandable and the second valve is self-expandable.
38. The method of statements 24-26, the first section of the catheter comprising a first balloon for expansion of the first valve and the third section of the catheter comprising a second balloon for expansion of the second valve, wherein the first valve is balloon expandable and the second valve is balloon expandable.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A valve assembly comprising:
a first valve;
a second valve;
a pouch connecting the first and second valve.

2. The valve assembly of claim 1, the first valve comprising a first leaflet and a second leaflet; and
the second valve comprising a first leaflet, a second leaflet, and a third leaflet.

3. The valve assembly of claim 1, further comprising a layer of adhesive on an outer surface of the prosthetic valve.

4. The valve assembly of claim 1, the pouch comprising:
a main compartment;
a first tubular section extending from the main compartment, the first tubular section attached to the outer surface of the first valve; and
a second tubular section extending from the main compartment, the second tubular section attached to the outer surface of the second valve.

5. The valve assembly of claim 4, the main compartment having a maximum size and volume equal to a size and volume of a heart ventricle.

6. The valve assembly of claim 1, wherein the first valve is configured to replace a native mitral valve and the second valve is configured to replace a native aortic valve.

7. The valve assembly of claim 1, wherein the first valve is configured to regulate flow of blood into the pouch, and the second valve is configured to regulate flow of blood out of the pouch.

8. The valve assembly of claim 4, the main compartment being tubular.

9. A delivery system comprising:
a catheter comprising a sheath defining a lumen and a shaft positioned inside the lumen of the sheath;
a valve assembly, the valve assembly positioned around the shaft and inside the sheath, the valve assembly comprising a first valve, a second valve, and a pouch connecting the first and second valves.

10. The delivery system of claim 9, the shaft of the catheter having a first section with a first diameter, a second section with a second diameter, and a third section with a third diameter where the second diameter is less than the first diameter rand the second diameter is less than the third diameter;
wherein the first valve is positioned around the first section, the pouch is positioned around the second section, and the second valve is positioned around the third section.

11. The delivery system of claim 9, wherein the pouch has a plurality of folds.

12. The delivery system of claim 11, wherein the plurality of folds are circumferential folds.

13. The delivery system of claim 11, wherein the plurality of folds are longitudinal folds.

14. The delivery system of claim 9, wherein the first valve comprises a first leaflet and a second leaflet; and
the second valve comprises a first leaflet, a second leaflet, and a third leaflet.

15. The delivery system of claim 9, the valve assembly further comprising a layer of adhesive on an outer surface of the valve assembly.

16. The delivery system of claim 9, wherein the first valve is configured to replace a native mitral valve and the second valve is configured to replace a native aortic valve.

17. The delivery system of claim 9, wherein the first valve is configured to regulate flow of blood into the pouch, and the second valve is configured to regulate flow of blood out of the pouch.

18. The delivery system of claim 9, the pouch being tubular.

19. The delivery system of claim 9, the pouch further comprising:
a main compartment;
a first tubular section extending from the main compartment, the first tubular section attached to the outer surface of the first valve; and
a second tubular section extending from the main compartment, the second tubular section attached to the outer surface of the second valve.

20. The delivery system of claim 20, the main compartment having a maximum size and volume equal to a size and volume of a heart ventricle.
